(19) United States
(12) Patent Application Publication
(10) Pub. No.: US 2020/0188162 A1
(43) Pub. Date: Jun. 18, 2020

(54) Ostomy Bag and Absorbent Insert Assembly

(71) Applicant: Julia Menifee, Las Vegas, NV (US)

(72) Inventor: Julia Menifee, Las Vegas, NV (US)

(21) Appl. No.: 16/218,784

(22) Filed: Dec. 13, 2018

(51) Int. Cl. A61F 5/445; A61F 5/441; A61F 5/44

(52) U.S. Cl. CPC A61F 5/445; A61F 5/455; A61F 5/4401; A61F 5/441

(57) ABSTRACT

An ostomy bag and absorbent insert assembly for odor and leak reduction includes a modified ostomy bag and an absorbent insert. The modified ostomy bag comprises a storage bag having a secondary aperture extending through to an inside. A closure is coupled to the storage bag to seal and alternatively unseal the secondary aperture. The absorbent insert comprises an outer layer. The outer layer is permeable. An gelatinizing agent is coupled within the outer layer and is configured to make urine gelatinous. The absorbent insert is insertable into, and removable from, the inside of the storage bag via the secondary aperture.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

### (2) Description of Related Art Including Information Disclosed Under 37 CFR 1.97 and 1.98

The disclosure and prior art relates to ostomy bags and more particularly pertains to a new ostomy bag for odor and leak reduction.

### (h) BRIEF SUMMARY OF THE INVENTION

An embodiment of the disclosure meets the needs presented above by generally comprising a modified ostomy bag and an absorbent insert. The modified ostomy bag comprises a storage bag having a secondary aperture extending through to an inside. A closure is coupled to the storage bag to seal and alternatively unseal the secondary aperture. The absorbent insert comprises an outer layer. The outer layer is permeable. An gelatinizing agent is coupled within the outer layer and is configured to make urine gelatinous. The absorbent insert is insertable into, and removable from, the inside of the storage bag via the secondary aperture.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front elevation view of an ostomy bag and absorbent insert assembly according to an embodiment of the disclosure.

FIG. 2 is a rear elevation view of an embodiment of the disclosure.

FIG. 3 is a cross-sectional view of an embodiment of the disclosure along line 3-3 of FIG. 2.

FIG. 4 is an isometric view of an embodiment of the disclosure.

### (j) DETAILED DESCRIPTION OF THE INVENTION

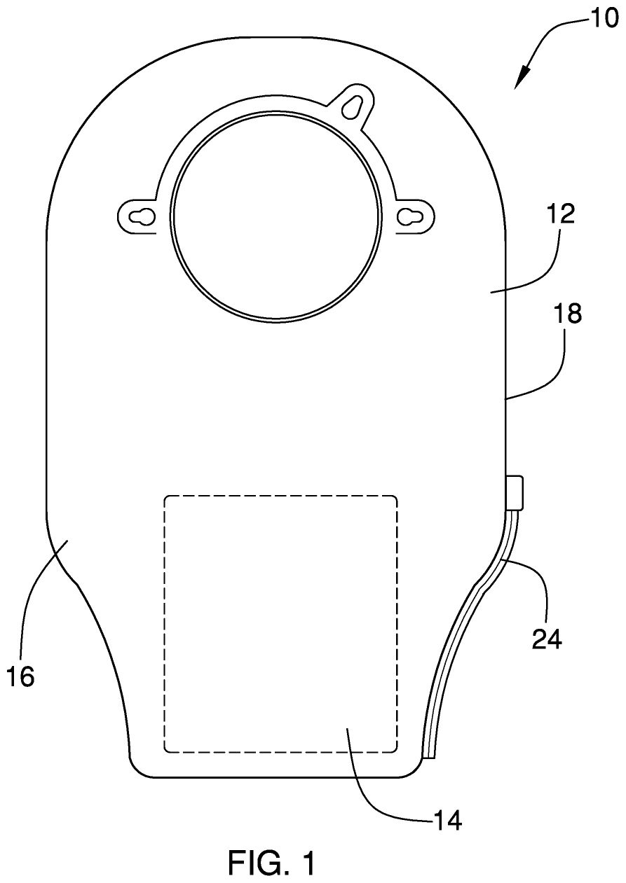

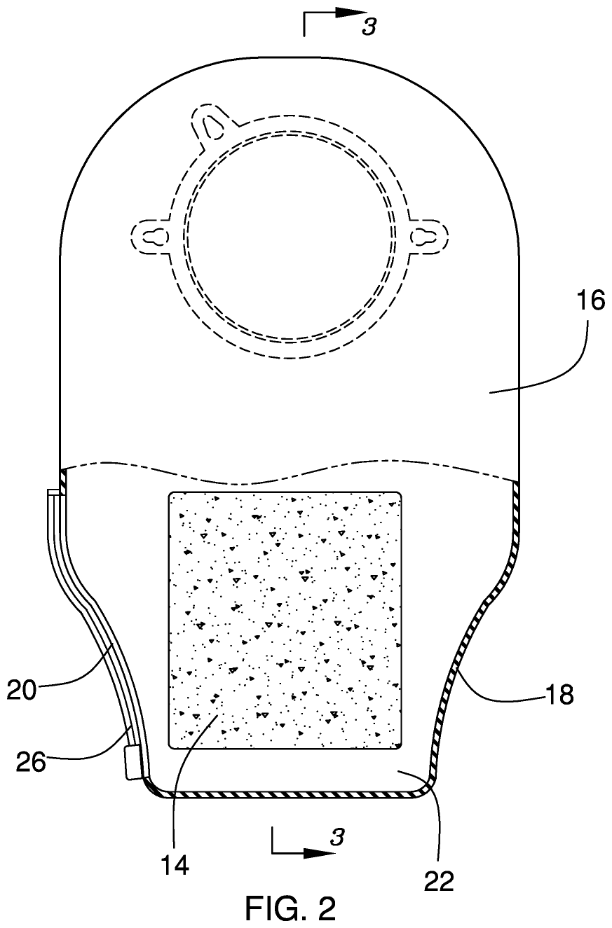

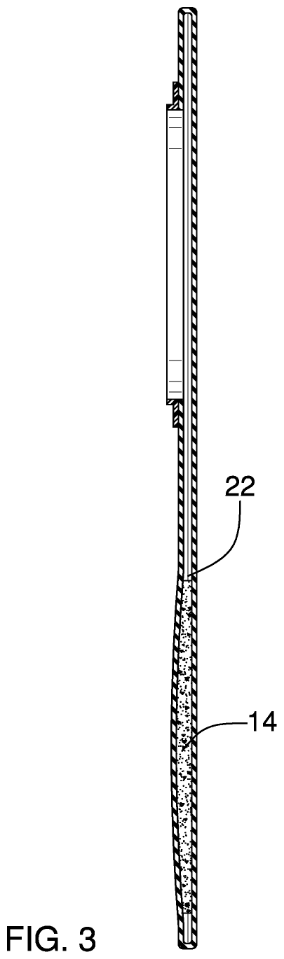

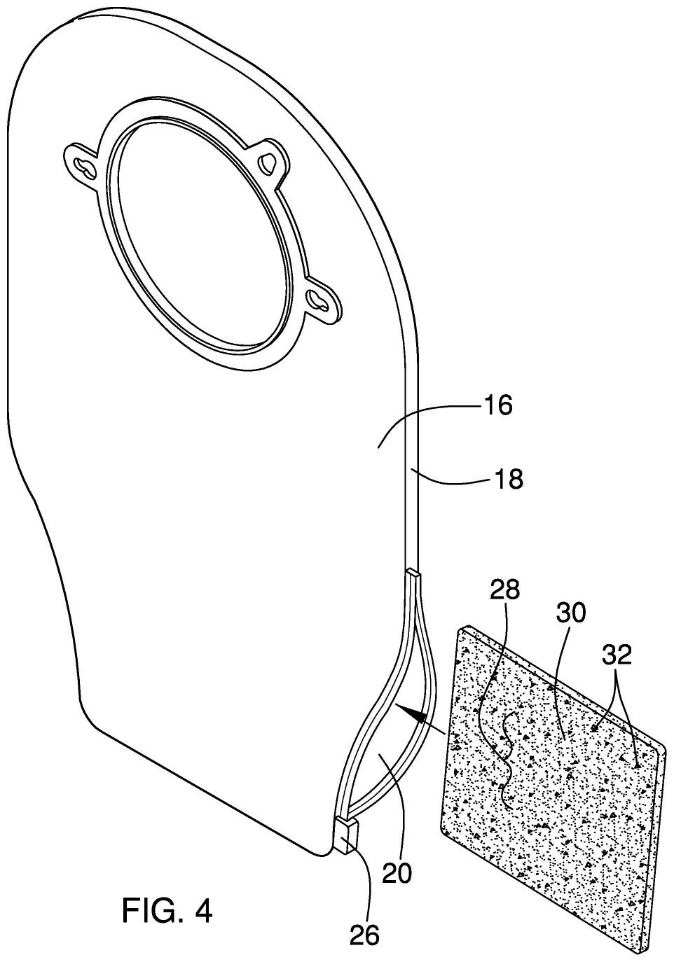

With reference now to the drawings, and in particular to FIGS. 1 through 4 thereof, a new ostomy bag embodying the principles and concepts of an embodiment of the disclosure and generally designated by the reference numeral 10 will be described.

As best illustrated in FIGS. 1 through 4, the ostomy bag and absorbent insert assembly 10 generally comprises a modified ostomy bag 12 and an absorbent insert 14. The modified ostomy bag 12 comprises a storage bag 16 having a perimeter 18. The storage bag 16 has a secondary aperture 20 extending through the perimeter 18 to an inside 22. A closure 24 is coupled to the storage bag 16. The closure 24 seals and alternatively unseals the secondary aperture 20. The closure 24 may be a zipper 26. The absorbent insert 14 comprises an outer layer 28. The outer layer 28 is permeable. An gelatinizing agent 30 is coupled within the outer layer 28 and is configured to make urine gelatinous. The absorbent insert 14 may also comprise a deodorizer 32 coupled within the outer layer 28. The absorbent insert 14 is insertable into, and removable from, the inside 22 of the storage bag via the secondary aperture 20.

In use, the absorbent insert 14 is placed within the inside 22 of the storage bag and the closure 24 is used to seal the secondary aperture 20. When a wearer urinates, the gelatinizing agent 30 turns the urine into a gelatinous consistency to prevent leakage. The deodorizer 32 aids in preventing smells from leaving the storage bag 16. The closure 24 may then be used to open the secondary aperture 20 to remove the soiled absorbent insert 14 and replace it.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An ostomy bag and absorbent insert assembly comprising:
a modified ostomy bag, the modified ostomy bag comprising:
a storage bag, the storage bag having a secondary aperture extending through to an inside; and
a closure coupled to the storage bag, the closure sealing and alternatively unsealing the secondary aperture; and
an absorbent insert, the absorbent insert comprising:
an outer layer, the outer layer being permeable; and
an gelatinizing agent, the gelatinizing agent being coupled within the outer layer, the gelatinizing agent being configured to make urine gelatinous;
wherein the absorbent insert is insertable into, and removable from, the inside of the storage bag via the secondary aperture.

2. The ostomy bag and absorbent insert assembly of claim 1 further comprising the absorbent insert comprising a deodorizer, the deodorizer being coupled within the outer layer.

3. The ostomy bag and absorbent insert assembly of claim 1 further comprising the closure being a zipper.

4. The ostomy bag and absorbent insert assembly of claim 1 further comprising the secondary aperture extending through a perimeter of the storage bag.

5. An ostomy bag and absorbent insert assembly comprising:
a modified ostomy bag, the modified ostomy bag comprising:
a storage bag, the storage bag having a perimeter, the storage bag having a secondary aperture extending through the perimeter to an inside; and
a closure coupled to the storage bag, the closure sealing and alternatively unsealing the secondary aperture, the closure being a zipper; and
an absorbent insert, the absorbent insert comprising:
an outer layer, the outer layer being permeable;
an gelatinizing agent, the gelatinizing agent being coupled within the outer layer, the gelatinizing agent being configured to make urine gelatinous; and
a deodorizer, the deodorizer being coupled within the outer layer;
wherein the absorbent insert is insertable into, and removable from, the inside of the storage bag via the secondary aperture.
